(19) United States
(12) Patent Application Publication
(10) Pub. No.: US 2018/0256137 A1
(43) Pub. Date: Sep. 13, 2018

(54) FLUID SENSING DEVICES WITH CONCENTRATION REGULATION

(71) Applicant: Eccrine Systems, Inc., Cincinnati, OH (US)

(72) Inventors: Jason Heikenfeld, Cincinnati, OH (US); Hector Wong, Cincinnati, OH (US); Kevin Plaxco, Santa Barbara, CA (US); Jacob A. Bertrand, Norwood, OH (US)

(73) Assignee: Eccrine Systems, Inc., Cincinnati, OH (US)

(21) Appl. No.: 15/769,435

(22) PCT Filed: Oct. 23, 2016
(86) PCT No.: PCT/US16/58357

§ 371 Date: Apr. 19, 2018

Related U.S. Application Data
(60) Provisional application No. 62/245,638, filed on Oct. 23, 2015.
(60) Provisional application No. 62/269,244, filed on Dec. 18, 2015.
(60) Provisional application No. 62/269,447, filed on Dec. 18, 2015.
(60) Provisional application No. 62/364,589, filed on Jul. 20, 2016.

(51) Int. Cl. A61B 10/00; G01N 33/487; A61B 5/145; A61B 5/1477; A61B 5/00; G01N 27/327; G01N 27/40

(52) U.S. Cl. CPC A61B 10/0064; G01N 33/48707; A61B 5/14521; A61B 5/14546; G01N 27/40; A61B 5/1477; A61B 5/6803; A61B 5/6804; G01N 27/3276; A61B 5/14539

(57) ABSTRACT

Embodiments of the disclosed invention provide devices and methods for buffering fluid samples to enable accurate concentration measurements of analytes by salinity-sensitive or pH-sensitive sensors. The buffering capabilities of the device include the ability to control the salinity and pH of a fluid sample, specifically, through the management of solutes such as salts, H⁺, other ions, and other solutes, that are found in sweat, biofluids, or other fluids. The purpose of such control is to enhance particular fluid sensing device applications by improving detectability of the targeted analyte, or improving performance of analyte sensors. Some embodiments also include components to enable sample concentration to enhance the measurement of low-concentration solutes found in the fluid.

### CROSS-REFERENCE TO RELATED APPLICATIONS

The present application claims priority to PCT/US2016/058357, filed Oct. 23, 2016; U.S. Provisional Application No. 62/364,589, filed Jul. 20, 2016; U.S. Provisional Application No. 62/245,638, filed Oct. 23, 2015; U.S. Provisional No. 62/269,244, filed Dec. 18, 2015; and U.S. Provisional Application No. 62/269,447, filed Dec. 18, 2015, the disclosures of which are hereby incorporated herein by reference in their entirety.

### BACKGROUND OF THE INVENTION

Non-invasive biosensing technologies have enormous potential for several medical, fitness, and personal well-being applications. The sweat ducts can provide a route of access to many of the same biomarkers, chemicals, and other solutes that are carried in blood and can provide significant information enabling one to diagnose ailments, health status, toxins, performance, and other physiological attributes even in advance of any physical sign. Sweat has many of the same analytes and analyte concentrations found in blood and interstitial fluid. Interstitial fluid has even more analytes nearer to blood concentrations than sweat does, especially for larger sized and more hydrophilic analytes (such as proteins).

However, one challenge for both fluids, especially for sweat, is that high-concentration ions such as Na⁺, K⁺, ammonium, Cl⁻, pH, and other chemical solutes in sweat can interfere with sensors specific to analytes such as aptamer sensors for cortisol, or amperometric/ion-selective sensors for urea. The primary issue is that the concentration of these interfering solutes can change over wide ranges. If such solutes were more stable in sweat, the resulting interference could be resolved through calibration or other suitable methods. One possible solution is to measure the solute concentrations in real-time, and to use those measurements to correct the other sensor readings. However, this solution inefficiently uses two sensors to achieve one sensing result, and compounds the individual errors from each sensor. What is needed are simple yet robust methods to chemically buffer a sweat, biofluid, or other fluid sample in a fluid sensing device, ideally without reducing chronologically assured sampling rates.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings biofluid sensing technology into proximity with a fluid as it is generated.

### SUMMARY OF THE INVENTION

Embodiments of the disclosed invention provide devices and methods for buffering fluid samples to enable accurate concentration measurements of analytes in the biofluid, or other fluid, by salinity-sensitive or pH-sensitive sensors. The buffering capabilities of the device include the ability to control the salinity and pH of a fluid sample, specifically, through the management of solutes in the fluid, such as salts, H⁺, other ions, and other fluid contents. The purpose of such control is to enhance particular fluid sensing device applications by improving detectability of the targeted analyte, or improving performance of analyte sensors. Some embodiments also include components to enable sample concentration to enhance the measurement of low-concentration fluid solutes.

### BRIEF DESCRIPTION OF THE DRAWINGS

The objects and advantages of the disclosed invention will be further appreciated in light of the following detailed descriptions and drawings in which:

FIG. 1 depicts an embodiment of the disclosed invention configured to provide buffering of fluid samples.

FIG. 2 depicts an embodiment of the disclosed invention configured to provide buffering of fluid samples.

FIG. 3 depicts an embodiment of the disclosed invention configured to provide buffering of fluid samples.

FIG. 4 depicts an embodiment of the disclosed invention configured to provide buffering of fluid samples.

### DEFINITIONS

As used herein, “sweat” or “sweat biofluid” means a biofluid that is primarily sweat, such as eccrine or apocrine sweat, and may also include mixtures of biofluids such as sweat and blood, or sweat and interstitial fluid, so long as advective transport of the biofluid mixtures (e.g., flow) is primarily driven by sweat.

As used herein, “biofluid” may mean any human biofluid, including, without limitation, sweat, interstitial fluid, blood, plasma, serum, tears, and saliva. A biofluid may be diluted with water or other solvents inside a device because the term biofluid refers to the state of the fluid as it emerges from the body.

As used herein, “fluid” may mean any human biofluid, or other fluid, such as water, including without limitation, groundwater, sea water, freshwater, etc., petroleum products, or other fluids.

As used herein, “continuous monitoring” means the capability of a device to provide at least one sensing and measurement of fluid collected continuously or on multiple occasions, or to provide a plurality of fluid measurements over time.

As used herein, “chronological assurance” is an assurance of the sampling rate for measurement(s) of sweat (or other biofluid or fluid), or solutes in sweat, being the rate at which measurements can be made of new sweat or its new solutes as they originate from the body. Chronological assurance may also include a determination of the effect of sensor function, or potential contamination with previously generated sweat, previously generated solutes, other fluid, or other measurement contamination sources for the measurement(s).

As used herein, “determined” may encompass more specific meanings including but not limited to: something that is predetermined before use of a device; something that is determined during use of a device; something that could be a combination of determinations made before and during use of a device.

As used herein, “sweat sampling rate” is the effective rate at which new sweat, or sweat solutes, originating from the sweat gland or from skin or tissue, reaches a sensor that measures a property of sweat or its solutes. Sweat sampling rate, in some cases, can be far more complex than just sweat generation rate. Sweat sampling rate directly determines, or is a contributing factor in determining, the chronological assurance. Times and rates are inversely proportional (rates having at least partial units of 1/seconds), therefore a short or small time required to refill a sweat volume can also be said to have a fast or high sweat sampling rate. The inverse of sweat sampling rate (1/s) could also be interpreted as a “sweat sampling interval(s)”. Sweat sampling rates or intervals are not necessarily regular, discrete, periodic, discontinuous, or subject to other limitations. Like chronological assurance, sweat sampling rate may also include a determination of the effect of potential contamination with previously generated sweat, previously generated solutes, other fluid, or other measurement contamination sources for the measurement(s). Sweat sampling rate can also be in whole or in part determined from solute generation, transport, advective transport of fluid, diffusion transport of solutes, or other factors that will impact the rate at which new sweat or sweat solutes reach a sensor and/or are altered by older sweat or solutes or other contamination sources. Sensor response times may also affect sampling rate.

As used herein, “sweat stimulation” is the direct or indirect causing of sweat generation by any external stimulus, the external stimulus being applied for the purpose of stimulating sweat. Sweat stimulation, or sweat activation, can be achieved by known methods. For example, sweat stimulation can be achieved by simple thermal stimulation, chemical heating pad, infrared light, by orally administering a drug, by intradermal injection of drugs such as carbachol, methylcholine or pilocarpine, and by dermal introduction of such drugs using iontophoresis. A device for iontophoresis may, for example, provide direct current and use large lead electrodes lined with porous material, where the positive pole is dampened with 2% pilocarpine hydrochloride and the negative one with 0.9% NaCl solution. Sweat can also be controlled or created by asking the device wearer to enact or increase activities or conditions that cause them to sweat. These techniques may be referred to as active control of sweat generation rate.

As used herein, “sweat generation rate” is the rate at which sweat is generated by eccrine sweat glands. Sweat generation rate is typically measured by the flow rate from each gland in nL/min/gland. In some cases, the measurement is then multiplied by the number of sweat glands from which sweat is being sampled to calculate the sweat volume sampled per unit time.

As used herein, “fluid sampling rate” is the effective rate at which new fluid, or fluid solutes, originating from the fluid source, reaches a sensor that measures a property of the fluid or its solutes. Fluid sampling rate directly determines, or is a contributing factor in determining, the chronological assurance. Times and rates are inversely proportional (rates having at least partial units of 1/seconds), therefore a short or small time required to refill a fluidic volume can also be said to have a fast or high fluid sampling rate. The inverse of fluid sampling rate (1/s) could also be interpreted as a “fluid sampling interval(s)”. Fluid sampling rates or intervals are not necessarily regular, discrete, periodic, discontinuous, or subject to other limitations. Like chronological assurance, fluid sampling rate may also include a determination of the effect of potential contamination with previously generated fluid, previously generated solutes, other fluid, or other measurement contamination sources for the measurement(s). Fluid sampling rate can also be in whole or in part determined from solute generation, transport, advective transport of fluid, diffusion transport of solutes, or other factors that will impact the rate at which new fluid or fluid solutes reach a sensor and/or are altered by older fluid or solutes or other contamination sources. Sensor response times may also affect sampling rate.

As used herein, “measured” can imply an exact or precise quantitative measurement and can include broader meanings such as, for example, measuring a relative amount of change of something. Measured can also imply a binary measurement, such as ‘yes’ or ‘no’, present/not present type measurements.

As used herein, “fluidic volume” is the fluidic volume in a space that can be defined multiple ways. Fluidic volume may be the volume that exists between a sensor and the point of generation of a fluid or a solute moving into or out of the fluid from the body or from other sources. Fluidic volume can include the volume that can be occupied by fluid between: the sampling site on the skin and a sensor on the skin, where the sensor has no intervening layers, materials, or components between it and the skin; or the sampling site on the skin and a sensor on the skin where there are one or more layers, materials, or components between the sensor and the sampling site on the skin.

As used herein, “solute generation rate” is simply the rate at which solutes move from the body or other sources into a fluid. “Solute sampling rate” includes the rate at which these solutes reach one or more sensors.

As used herein, “microfluidic components” are channels in polymer, textiles, paper, or other components known in the art of microfluidics for guiding movement of a fluid or at least partial containment of a fluid.

As used herein, “state void of fluid” means a fluid sensing device component, such as a space, material or surface, that can be wetted, filled, or partially filled by fluid, when the component is entirely or substantially (e.g., >50%) dry or void of fluid.

As used herein, “advective transport” is a transport mechanism of a substance, or conserved property by a fluid, that is due to the fluid's bulk motion.

As used herein, “diffusion” is the net movement of a substance from a region of high concentration to a region of low concentration. This is also referred to as the movement of a substance down a concentration gradient.

As used herein, a “sample concentrator” is any portion of a device, material, subsystem, or other component that can be utilized to increase the molarity of at least one fluid analyte, at least in part by removing a portion of the water that was originally with the at least one analyte when it exited the body.

As used herein, the term “analyte-specific sensor” is a sensor that performs specific chemical recognition of an analyte's presence or concentration (e.g., ion-selective electrodes, enzymatic sensors, electrochemical aptamer-based sensors, etc.). For example, sensors that sense impedance or conductance of a fluid, such as sweat, are excluded from the definition of analyte-specific sensor because sensing impedance or conductance merges measurements of all ions in sweat (i.e., the sensor is not chemically selective; it provides an indirect measurement). Sensors could also be optical, mechanical, or use other physical/chemical methods which are specific to a single analyte. Further, multiple sensors can each be specific to one of multiple analytes.

“EAB sensor” means an electrochemical aptamer-based biosensor that is configured with multiple aptamer sensing elements that, in the presence of a target analyte in a fluid sample, produce a signal indicating analyte capture, and which signal can be added to the signals of other such sensing elements, so that a signal threshold may be reached that indicates the presence or concentration of the target analyte. Such sensors can be in the forms disclosed in U.S. Pat. Nos. 7,803,542 and 8,003,374 (the “Multi-capture Aptamer Sensor” (MCAS)), or in U.S. Provisional Application No. 62/523,835 (the “Docked Aptamer Sensor” (DAS)).

As used herein, “sample volume” is the fluidic volume in a space that can be defined multiple ways. Sample volume may be the volume that exists between a sensor and the point of generation of a fluid sample. Sample volume can include the volume that can be occupied by sample fluid between: the sampling site on the skin and a sensor on the skin where the sensor has no intervening layers, materials, or components between it and the skin; or the sampling site on the skin and a sensor on the skin where there are one or more layers, materials, or components between the sensor and the sampling site on the skin.

As used herein, a “volume-reduced pathway” or “reduced-volume pathway” is at least a portion of a sample volume that has been reduced by addition of a material, device, layer, or other component, which therefore increases the sampling interval for a given sample generation rate. A volume-reduced pathway can be created by at least one volume reducing component.

As used herein, “buffering component” is any component that regulates concentration of at least one chemical in the collected sample preferably within at least 20% of a target concentration of at least one chemical, and less preferably within at least 100% or at least 300% of a target concentration.

### DETAILED DESCRIPTION OF THE INVENTION

Embodiments of the disclosed invention apply at least to any type of fluid sensing device that measures at least one analyte in sweat, interstitial fluid, other biofluid, or other fluid. Further, embodiments of the disclosed invention apply to sensing devices which measure samples at chronologically assured sampling rates or intervals. Further, embodiments of the disclosed invention apply to sensing devices which can take on forms including patches, bands, straps, portions of clothing, wearables, or any suitable mechanism that reliably brings sampling and sensing technology into intimate proximity with a fluid sample as it is transported to the skin surface. While some embodiments of the disclosed invention utilize adhesives to hold the device near the skin, devices could also be held by other mechanisms that hold the device secure against the skin, such as a strap or embedding in a helmet. Certain embodiments of the disclosed invention show sensors as simple individual elements. It is understood that many sensors require two or more electrodes, reference electrodes, or additional supporting technology or features which are not captured in the description herein. Sensors are preferably electrical in nature, but may also include optical, chemical, mechanical, or other known biosensing mechanisms. Sensors can be in duplicate, triplicate, or more, to provide improved data and readings. Certain embodiments of the disclosed invention show sub-components of what would be sensing devices with more sub-components needed for use of the device in various applications, which are obvious (such as a battery), and for purposes of brevity and of greater focus on inventive aspects, such components are not explicitly shown in the diagrams or described in the embodiments of the disclosed invention.

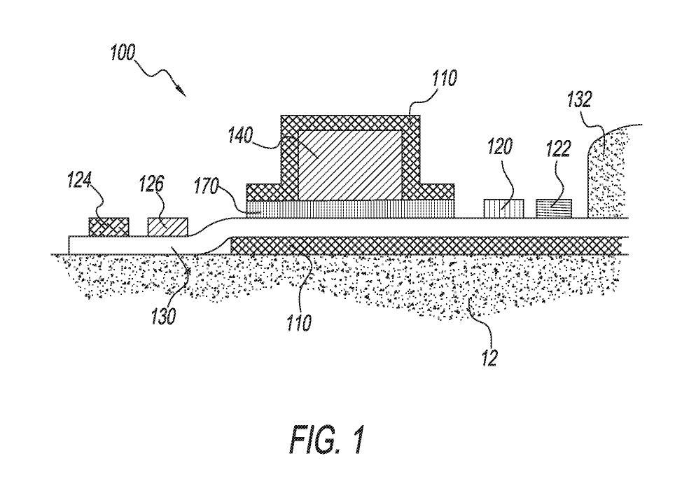

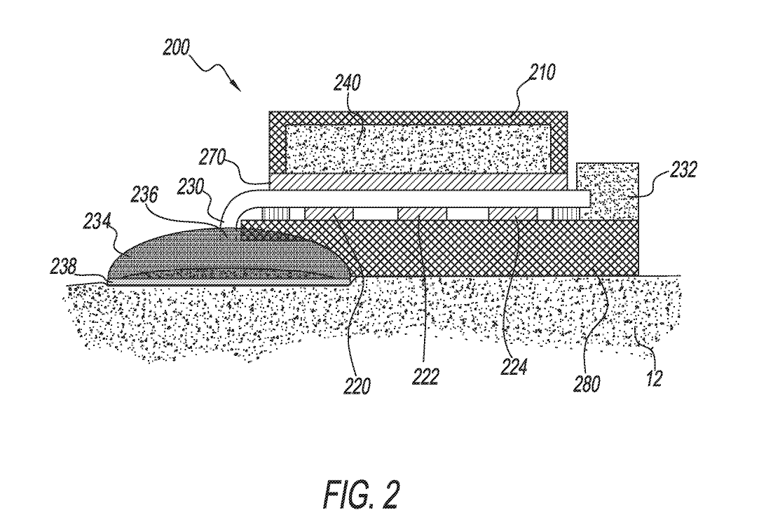

With reference to FIG. 1, in a disclosed embodiment, at least a portion of a fluid sensing device 100 is shown and positioned on the skin 12. The device 100 includes one or more primary sensors 120, 122, and may also include one or more reference sensors 124, 126. The device further includes a polymer substrate 110 and polymer casing 110 made of PET or other suitable material. A fluid collector 130 carries sweat from skin 12 to sensors 120, 122, 124, 126, and onto a fluid sample pump 132 by any suitable mechanism for transport, including osmosis, positive pressure from sweat ducts, or wicking pressures. In some embodiments, the collector 130 and sample pump 132 could be paper or textile wicks. In some embodiments, the collector 130 may include separate collection and transport components (see, e.g., FIG. 2). For example, the fluid collector 130 may include a microchannel that is in fluidic communication with a sample collection component, sensors and sample pump (not shown). The sample pump 232 may be comprised of a desiccant, a sponge, a hydrogel, or other suitable material capable of drawing a fluid sample through the fluid collector, and/or absorbing fluid after it has interacted with the sensors. The device further includes a chemical buffering fluid, gel, solid, or other material 140 and a membrane 170 which, along with casing 110, forms a buffering component.

In some embodiments, the fluid collector 130 itself may perform sample buffering, and in such configurations, a separate buffering component may not be required. The buffering fluid collector may be impregnated with buffering chemicals, or may be chemically modified to provide buffering to the fluid sample as it passes through the fluid collector. For example, the fluid collector could include an ion exchange resin, which would be configured to reduce the concentration of ions that could interfere with the particular measurements needed for a fluid sensing device application. In other embodiments, the buffering fluid collector may be used in combination with a separate buffering component.

In an example embodiment, the primary sensors 120 and 122 are electrochemical aptamer-based (“EAB”) sensors for hormones, which responses will vary with, for example, changes in fluid concentrations of Na⁺ and Cl⁻ (salinity), and H⁺ (pH). For a more complete discussion of EAB sensor variance with salinity and pH, see PCT/US17/23399, which is hereby incorporated by reference herein in its entirety. The buffering component contains, for example, a buffering fluid having 40 mM Na⁺, a pH of 7, and has a fluid volume that is at least one hundred times, or at least one thousand times, or at least ten thousand times greater than the fluid volume of fluid collector 130, e.g., a buffering component volume of 10 μL. In various embodiments, the buffering component may contain a reagent, NaCl, KCl, pH, urea, ammonia, lactate, a reference analyte, or a target analyte. Membrane 170 could be, for example, a PVC polymer membrane embedded with ionophores for Na⁺, Cl⁻, and pH, or just one of these, such that the membrane is relatively impermeable but is more permeable to the chemicals to be buffered. In some embodiments, membrane 170 may be a dialysis membrane. Due to diffusion across the membrane, the fluid sample salinity is stabilized at around 30 mM and pH is stabilized near 7, as the fluid sample reaches the primary sensors 120, 122. In other embodiments, the device may include additional buffering components, each with one or more chemicals and membranes.

With further reference to FIG. 1, several enhancements are possible for device 100. The reference sensors 124, 126, could be analyte-specific sensors for the fluid solutes to be buffered. For example, if the buffering component were imperfect at regulating concentrations in some circumstances (e.g., at very high sweat rates) then the reference sensors 124, 126 could be used to correct for variations in the primary sensors 120, 122 caused by the buffering of the solutes. A biofluid such as sweat also contains many other chemical constituents. If such constituents are not in the buffering component, then water (if that is the fluid in the buffering component) would favor transport by osmosis out of the buffering component and into the sensing area. Therefore, in the disclosed invention, the buffering component may contain an artificial fluid, such as an artificial sweat formulation, that contains concentrations of a plurality of analytes to mitigate such osmosis.

In another embodiment, the disclosed invention may combine the buffering component with a sample concentration component. For further description of sample concentration devices and methods, see PCT/US16/58356, which is hereby incorporated by reference herein in its entirety. In some embodiments, a sample concentration component and a buffering component could be the same component. For example, with reference to FIG. 2, where like numerals refer to like features of previous figures, a device 200 of the disclosed invention is built upon a substrate 280. The device includes a combined buffering and sample concentration component 210, that may include a forward osmosis membrane 270 for concentrating a sample with respect to a target analyte (e.g., cortisol), and a buffering concentrator (“BC”) solution or material 240, which may be, e.g., a disaccharide. The BC solution 240 also contains a 20 mM concentration of NaCl and is at a pH of 7. The membrane 270 allows NaCl and H⁺ to flow freely through, therefore regulating the NaCl and pH in the fluid sample as it flows from skin 12, through fluid microchannel 230 to the sensors 220, 222, 224 and into the fluid sample pump 232. In various embodiments, the device may regulate the concentration of a solute in the fluid sample to within at least 20%, to within at least 100%, or to within at least 300% of a target concentration of the solute.

With further reference to FIG. 2, an embodiment of the device includes a sweat collector 234 having a determined area of contact with a wearer's skin 12. The sweat collector 234 presents a concave shape toward the skin, and has sufficient clearance from skin so that when the device is worn on the body sweat can flow into the device at a natural sweat rate. When applied to a wearer's skin, some of the skin will bulge into the collection area, which aids in providing a seal with skin, but also potentially occludes sweating if the collector is allowed to apply pressure to the sweat ducts. See Johnson, C., et al., “The use of partial sweat duct occlusion in the elucidation of sweat duct function in health and disease,” J. Soc. Cosmet. Chem. 24 15-29 (1973). Some embodiments may include internal ridges (not shown) to maintain space for sweat to flow to the inlet 236. The inlet 236 is in fluidic communication with the microchannel 230, and hence the sensors 220, 222, 224, and sample pump 232. The sweat collector also includes a flexible sealing component 238, which is, for example, a latex or rubber o-ring, a screen-printed silicone gasket, flexible injection-molded ridge, or other suitable material. The sealing component 238 is configured to prevent sweat entering the collection area from the surrounding skin, and to reduce contamination from the skin surface when the device shifts position during normal wear.

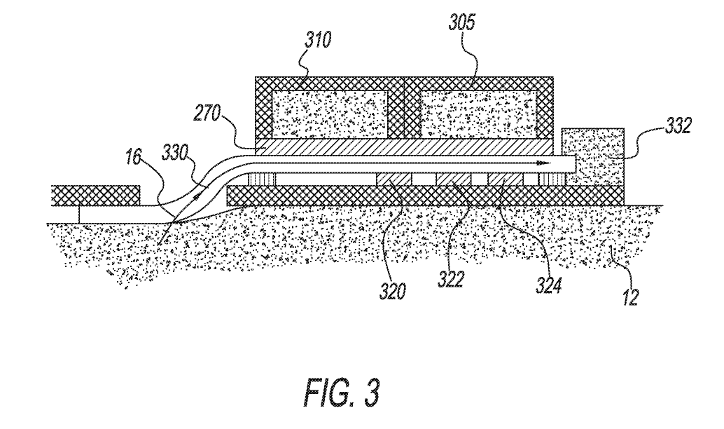

With reference to FIG. 3, where like numerals refer to like features of previous figures, in a preferred embodiment, the buffering component 305 is located after the sample concentration component 310 in relation to the flow direction of the fluid sample 16, so that the fluid is concentrated first, and buffered second. This is because many sample concentration component embodiments could increase salinity, change pH, or concentrate larger acids, bases, or other chemicals that could distort sensor signals. In other embodiments, a buffering component could be configured before a sample concentration component in relation to the flow of the fluid sample, so that the fluid sample is buffered first and concentrated second. In this embodiment, the buffering component could establish a known concentration, such as salinity, which would allow the sample concentration component to have a more predictable degree of sample concentration. For example, a buffering component could regulate the concentration of NaCl to 20 mM and the sample concentration component could have 200 mM draw solution to therefore create a more predictable 10× concentration of the fluid sample before it reaches the analyte-specific sensors.

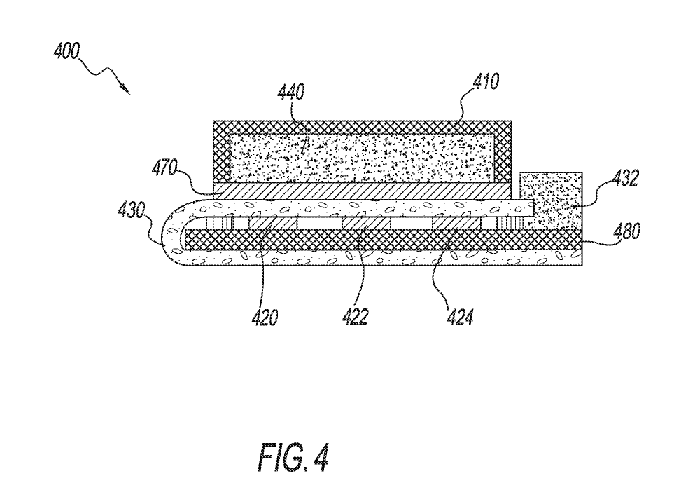

With reference to FIG. 4, where like numerals refer to like features of previous figures, a device 400 of the disclosed invention that is configured for use in a fluid sample is depicted. Rather than a sweat collector for placing in contact with skin, the device instead has a fluid collector 430 that is constructed of, e.g., sponge material. In this configuration, the fluid collector 430 could be placed in a container of drinking water (not shown). The device includes a buffering component 410, that may include a forward osmosis membrane 470 for concentrating a sample with respect to a target analyte (e.g., Cryptosporidium or one of that organism's products or toxins), and a buffering solution or material 440, which may contain a 20 mM concentration of NaCl and is at a pH of 7. The membrane 470 allows NaCl and pH to flow freely through, therefore regulating the NaCl and pH in the fluid sample as it is absorbed from the drinking water container, flows through fluid collector 430 to the sensors 420, 422, 424, and then into the fluid sample pump 432.

This has been a description of the disclosed invention along with a preferred method of practicing the invention, however the invention itself should only be defined by the appended claims.

What is claimed is:

1. A sensing device for chemically buffering a fluid sample, the sensing device comprising:
one or more primary sensors for measuring a characteristic of a target analyte in the fluid sample;
a fluid collector in fluidic communication with a fluid source and the one or more primary sensors, wherein the fluid collector is configured to transport the fluid sample to the one or more primary sensors; and
one or more buffering components in fluidic communication with the fluid collector, the one or more buffering components configured to adjust one or more solutes in the fluid sample, wherein the one or more buffering components include a selectively porous membrane that is porous to said one or more solutes.

2. The sensing device of claim 1, further comprising: one or more secondary sensors configured to measure a characteristic of a reference analyte in the fluid sample.

3. The sensing device of claim 1, further comprising: a fluid sample pump in fluidic communication with the fluid collector.

4. The sensing device of claim 1, wherein the fluid collector includes a buffering component.

5. The sensing device of claim 4, wherein the fluid collector includes an ion exchange resin.

6. The sensing device of claim 1, wherein the one or more buffering components are configured to adjust an initial concentration of a solute of the one or more solutes in the fluid sample to a final concentration that is greater than 50% and less than 200% of the initial concentration of the solute in the fluid sample.

7. The sensing device of claim 1, including a sweat stimulation component configured to induce the fluid sample.

8. The sensing device of claim 1, wherein the fluid sample includes sweat, interstitial fluid, blood, or saliva.

9. The sensing device of claim 1 wherein the one or more buffering components include a fluid volume that is at least a hundred times, at least a thousand times, or at least ten thousand times greater than a fluid volume of a fluid sample adjacent to the one or more buffering components.

10. The sensing device of claim 1, further comprising:
a sample concentrator configured to generate a concentrated form of the fluid sample to increase a first molarity of the target analyte within the fluid sample to a second molarity, wherein the second molarity is higher than the first molarity.

11. The sensing device of claim 10, wherein the one or more buffering components and the sample concentrator use a same selectively porous membrane.

12. The sensing device of claim 10, wherein the one or more buffering components are positioned relative to the sample concentrator to cause the fluid sample to be buffered prior to being concentrated.

13. The sensing device of claim 10, wherein the one or more buffering components are positioned relative to the sample concentrator to cause the fluid sample to be concentrated prior to being buffered.

14. A method of using a sensing device configured for fluid sample buffering, the method comprising:
receiving a fluid sample, wherein the fluid sample is received upon being in fluidic communication with the sensing device;
buffering the fluid sample with respect to one or more solutes;
receiving, using one or more sensors, a measurement indicative of a characteristic of a target analyte in the fluid sample; and
correlating the measurement with a physiological condition associated with a biological source of the fluid sample.

15. The method of claim 14, further comprising using a sweat stimulation component to stimulate sweat from the biological source.

16. The method of claim 14, further comprising:
receiving, using the one or more sensors, a measurement indicative of a characteristic of a reference analyte in the fluid sample;
comparing the measurement indicative of the characteristic of the reference analyte to the measurement indicative of the characteristic of the target analyte; and
determining, based on comparing the measurement indicative of the characteristic of the reference analyte to the measurement indicative of the characteristic of the target analyte, a change in a concentration of the target analyte in the fluid sample.

17. The method of claim 14, further comprising: concentrating the fluid sample with respect to the target analyte.

18. The method of claim 17, wherein the fluid sample is concentrated prior to being buffered.

19. The method of claim 17, wherein the fluid sample is buffered prior to being concentrated.

20. The method of claim 14, wherein a concentration of the target analyte in the fluid sample is within one of the following ranges: at least 20% of a target concentration, at least 100% of the target concentration, or at least 300% of the target concentration.
